(19) United States
(12) Patent Application Publication
(10) Pub. No.: US 2022/0143106 A1
(43) Pub. Date: May 12, 2022

(54) TREATMENT OF CLOSTRIDIUM DIFFICILE INFECTION WITH SPECIFIC CARBOHYDRATE DIET

(71) Applicant: Seattle Children's Hospital d/b/a Seattle Children's Research Institute, Seattle, WA (US)

(72) Inventor: David Suskind, Seattle, WA (US)

(73) Assignee: Seattle Children's Hospital d/b/a Seattle Children's Research Institute, Seattle, WA (US)

(21) Appl. No.: 17/428,375

(22) PCT Filed: Feb. 14, 2020
(86) PCT No.: PCT/US2020/018366

§ 371 Date: Aug. 4, 2021

Related U.S. Application Data
(60) Provisional application No. 62/806,578, filed on Feb. 15, 2019.
(60) Provisional application No. 62/811,439, filed on Feb. 27, 2019.

(51) Int. Cl. A61K 35/741; A23L 33/00; A23L 33/135

(52) U.S. Cl. CPC A61K 35/741; A23L 33/135; A23L 33/30

(57) ABSTRACT

Methods of reducing or inhibiting Clostridium infection, treating intestinal dysbiosis associated with Clostridium infection, and preventing Clostridium colonization or Clostridium recolonization in a subject are disclosed. The methods include administering to the subject a whole foods exclusionary diet, such as a diet that excludes grains, sugars other than honey, and milk products other than hard cheeses and yogurt fermented for greater than 24 hours.

### CROSS-REFERENCE TO RELATED APPLICATIONS

This application claims the benefit of U.S. Provisional Application No. 62/806,578 filed Feb. 15, 2019, and U.S. Provisional Application No. 62/811,439 filed Feb. 27, 2019, the disclosures of which are incorporated herein in their entirety.

### BACKGROUND

Clostridium difficile is an important nosocomial pathogen in adults and children. Roughly 4-5% of non-hospitalized healthy adults carry the organism in their intestinal flora, while adults in long term care facilities have asymptomatic carriage rates estimated at 20-50%. Clostridium difficile colonization results in a spectrum of clinical conditions from asymptomatic carrier state to fulminant colitis. Changes in the fecal microbiome are central in the development of C. difficile colonization and disease pathogenesis. Clostridium difficile infection has been shown to be associated with reduced biodiversity of the gut microbiome and intestinal dysbiosis. Klebsiella pneumoniae and Ruminococcus gnavus were noted to be associated with C. difficile carriage in an infant study.

Understanding the mechanism by which C. difficile is established within the gastrointestinal tract is vital to effective prevention, control, and therapy of C. difficile infection and conditions associated therewith. However, despite the advances of the art, a need remains for effective strategies that can simultaneously reduce or inhibit C. difficile in a subject while promoting development of healthy and diverse microbiota. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, provided herein is a method of treating intestinal dysbiosis associated with Clostridium infection in a subject, comprising administering to the subject in need thereof a whole foods exclusionary diet for an effective time, wherein the whole foods exclusionary diet is a diet that excludes grains, sugars other than honey, and milk products other than hard cheeses and yogurt fermented for greater than 24 hours.

In another aspect, provided herein is a method of preventing Clostridium colonization or Clostridium recolonization in the intestines of a subject, comprising administering to the subject in need thereof a whole foods exclusionary diet for an effective time, wherein the whole foods exclusionary diet is a diet that excludes grains, sugars other than honey, and milk products other than hard cheeses and yogurt fermented for greater than 24 hours.

In another aspect, provided herein is a method of reducing or inhibiting Clostridium infection in a subject, comprising administering to the subject in need thereof a whole foods exclusionary diet for an effective time, wherein the whole foods exclusionary diet is a diet that excludes grains, sugars other than honey, and milk products other than hard cheeses and yogurt fermented for greater than 24 hours.

In some embodiments, the Clostridium is Clostridium difficile.

In some embodiments, the subject is a mammal, e.g., a human. In some embodiments, the subject is a human child 18 years old or less or a human adult.

In some embodiments, the whole foods exclusionary diet is administered for at least one week, at least two weeks, at least three weeks, at least 1 month, at least 2 months, or at least 3 months. In some embodiments, the effective time of administration of the whole foods exclusionary diet is at least one week, at least two weeks, at least three weeks, at least 1 month, at least 2 months, or at least 3 months.

In some embodiments, the subject has been treated with one or more antibiotics effective against Clostridium prior to administration of the whole foods exclusionary diet.

In some embodiments, the whole foods exclusionary diet is a nutritionally complete grain-free diet with low sugar and lactose content. In some embodiments, the whole foods exclusionary diet is a nutritionally complete diet that excludes grains, grain products, milk products other than 24-hour fermented homemade yogurt and cheeses aged greater than 30 days, starchy vegetables, root vegetables, processed foods, and added sweeteners other than honey. In some embodiments, the whole foods exclusionary diet is Specific Carbohydrate Diet™.

In some embodiments, the whole foods exclusionary diet comprises one or more of the following: meats without additives, poultry, fish, shellfish, eggs; legumes, including dried navy beans, lentils, peas, split peas, unroasted cashews and peanuts in a shell, all-natural peanut butter, lima beans; hard cheeses aged more than 30 days, homemade yogurt fermented for at least 24 hours; fresh, frozen, non-starchy vegetables, string beans, whole fruits, whole nuts, whole nut flours, vegetable oils, teas, coffee, mustard, cider vinegar, white vinegar, juices with no additives, and honey.

In some embodiments, the whole foods exclusionary diet lacks or has minimal amount of calories derived from one or more of the following: added sugars, molasses, maple syrup, agave syrup, sucrose, processed fructose, high-fructose corn syrup, grains, lactose, starch derived from tubers, pectin, sugar alcohols, and fructooligosaccharides (FOS). In some embodiments, the whole foods exclusionary diet lacks or has minimal amount of calories derived from one or more of the following: corn, wheat, wheat germ, barley, oats, rice, bread, pasta, baked goods made with grain-based flour; seaweed, seaweed byproducts, potatoes, sweet potatoes, turnips; canned meats, processed meats, canola oil, commercial mayonnaise; milk, milk products high in lactose, soft cheese, commercial yogurt, cream, sour cream, ice cream, candy, and chocolate. In some embodiments, the minimal amount of calories is about 5% or less, about 4% or less, about 3% or less, about 2% or less, or about 1% or less of total dietary calories.

### DETAILED DESCRIPTION

The detailed description set forth below is intended as a description of various embodiments of the disclosed subject matter and is not intended to represent the only embodiments. Each embodiment described in this disclosure is provided merely as an example or illustration and should not be construed as preferred or advantageous over other embodiments. The illustrative examples provided herein are not intended to be exhaustive or to limit the claimed subject matter to the precise forms disclosed.

The present disclosure generally relates to treating Clostridium difficile infections in a subject. As described in more detail below, a single center, open diet-controlled study was performed. Patients with persistent C. difficile colonization were enrolled from outpatient clinics at Seattle Children's Hospital. Participants were provided meals prepared by a chef knowledgeable in the SCD™. Patients also received a list of food appropriate for the SCD™. Patients maintained on the SCD for one month and then liberalized to a heart healthy whole foods diet. Clinical evaluation occurred at baseline, 2, 4, and 12 weeks. Physician evaluation, anthropometrics, and a dietician visit occurred during each study visit. Stool PCR for C. difficile antigen/toxin and microbiome analysis occurred at each visit. Four participants enrolled: one female and three males, ages 2 years to 16 years old. Underlying diagnoses included Hurler syndrome status post bone marrow transplant (BMT), Crohn's disease, and Pre-B cell acute lymphoblastic leukemia (ALL) status post BMT. One participant was excluded due to negative C. difficile testing at screening. The three remaining participants completed the study. One participant had negative C. difficile infection by stool antigen/toxin testing starting at week 2. Another participant experienced clearance by week 4. Both participants remained negative through week 12. The third participant did not have C. difficile clearance, although he did report more formed stools. There were no adverse effects reported in this study. The study demonstrated that a whole foods exclusionary diet, such as the SCD™ diet, was safe and efficacious in human subjects with C. difficile colonization, e.g., pediatric patients with C. difficile colonization.

In accordance with the foregoing, in one aspect, the disclosure provides a method of reducing or inhibiting Clostridium infection in a subject. The method comprises modifying the subject's diet to remove all (or substantially decrease) intake of grains, milk products (except for hard cheeses and yogurt fermented at least 24 hours), and sweeteners such as added sugars (except for honey whole). As used herein, the diet of the methods can also be referred to a whole foods exclusionary diet, such as Specific Carbohydrate Diet™ (SCD™). The administration of the diet of the disclosure, such as whole foods exclusionary diet is maintained for a time effective to reduce or inhibit Clostridium infection in the subject. As used herein, the term “reducing” refers to a measurable reduction in Clostridium bacteria in the subject, such as statistically significant reduction. In some embodiments, a reduction means lowering the amount of bacteria detected by about 10%, by about 20%, by about 30%, by about 40%, by about 50%, by about 60%, by about 70%, by about 80%, or by about 90% as compared to pre-treatment. The amount of bacteria can be measured from the subject's gut flora, for example from stool samples. The amount can be specifically quantified by, for example, PCR-based detection methods such quantitative PCR (qPCR). The term “inhibiting Clostridium infection” refers to preventing an increase in Clostridium bacteria. The amount can be measured in absolute metrics or as a proportion to one or more other members of the subject's micro-flora in the intestines. For example, in one embodiment, the method of inhibiting Clostridium infection results in a stable or decreased proportion of Clostridium relative to the overall micro-flora content of the intestines.

In another aspect, the disclosure provides a method of preventing Clostridium colonization or recolonization in a subject's intestines. The method comprises modifying the subject's diet to remove all (or substantially decrease) intake of grains, milk products (except for hard cheeses and yogurt fermented at least 24 hours), and sweeteners such as added sugars (except for honey whole). This can be referred to a whole foods exclusionary diet. The administration of the whole foods exclusionary diet is maintained for a time effective to prevent Clostridium infection in the subject. The term prevention encompasses providing for a lack of detectable Clostridium in the subject's intestines during the administration of the whole foods exclusionary diet. In some embodiments, the method provides for a lack of detectable Clostridium in the subject's intestines for a discrete period of time following the administration of the whole foods exclusionary diet. The discrete period of time can be 1 week, 2 weeks, 3 weeks, 4 weeks, 5 weeks, 6 weeks, 7 weeks, 8 weeks, 9 weeks, 10 weeks, 3 months, 4 months, 5 months, or more beyond the administration of the whole foods exclusionary diet.

In another aspect, the disclosure provides a method of treating intestinal dysbiosis associated with Clostridium infection in a subject. The method comprises modifying the subject's diet to remove all (or substantially decrease) intake of grains, milk products (except for hard cheeses and yogurt fermented at least 24 hours), and sweeteners (except for honey whole). This can be referred to a whole foods exclusionary diet.

As used herein, the term “treat” refers to medical management of a disease, disorder, or condition (e.g., intestinal dysbiosis as described herein) of a mammalian subject (e.g., a human or non-human mammal, such as another primate, horse, dog, mouse, rat, guinea pig, rabbit, and the like). Treatment can encompass any indicia of success in the treatment or amelioration of the disease or condition (e.g., intestinal dysbiosis), including any parameter such as abatement, remission, diminishing of symptoms or making the disease or condition more tolerable to the patient, slowing in the rate of degeneration or decline, or making the degeneration less debilitating. Specifically in the context of dysbiosis, the term treat can encompass slowing or inhibiting the rate of Clostridium infection, or reducing the likelihood of recolonization, compared to not having the treatment. In some embodiments, the treatment encompasses resulting in some detectable degree reduction in Clostridium infection or Clostridium toxin in the patient. The amount of bacteria can be specifically quantified by, for example, PCR-based detection methods such quantitative PCR (qPCR) as described above. In some embodiments, the treatment encompasses resulting in some detectable increase of commensal diversity in the micro-flora in the subject's intestines. The treatment or amelioration of symptoms can be based on objective and/or subjective parameters, including the results of an examination by a physician. Accordingly, the term “treating” includes the modification of the subject's diet to alleviate, or to arrest, or to inhibit development of the symptoms or conditions associated with disease or condition (e.g., dysbiosis). The term “therapeutic effect” refers to the amelioration, reduction, or elimination of the disease or condition, symptoms of the disease or condition, or side effects of the disease or condition in the subject. The method can be conducted for a time effective to ameliorate the symptoms of dysbiosis.

In another aspect, the disclosure provides compositions for use in reducing or inhibiting Clostridium infection in a subject, preventing Clostridium colonization in a subject's intestines, or preventing Clostridium recolonization in a subject's intestines. The compositions comprise dietary foods, such as foods of a whole foods exclusionary diet, that are administered to a subject in need thereof. The dietary foods optionally comprise any “allowed” food and ingredient listed below. Furthermore, the dietary foods exclude the foods and ingredients “not allowed” as listed below. In some embodiments, the compositions comprise SCD™.

In any of the aspects referred to above, the step of modifying the subject's diet, including administering a diet encompasses both direct provision of food and meals according to the modified diet as well as prescribing the patient a dietary modification, including instructing the patient or patient's caregiver to conform to the limitations of the diet modification (e.g., whole foods exclusionary diet such as SCD™, as described below).

In any of the aspects referred to above, modifying the subject's diet to substantially decrease intake of grains, milk products, and sweeteners refers to a near complete eradication of such ingredients from the subject's diet. This allows for some residual or minor component of the diet to include the excluded components, but only to a degree that has no measurable effect on the results described herein. For example, such the indicated residual or minor component of the diet can result in less than about 5%, less than about 4%, less than about 3%, less than about 2% or less than about 1% of the total calories of the diet being attributed to the excluded components. In some embodiments, the excluded components such as grains, added sweeteners other than honey, (e.g., sugar), and milk products (other than hard cheeses or yogurts fermented for 24 hours or longer), comprise a minimal portion of the total calories consumed by the subject. In some embodiments, the minimal portion of calories is about 5% or less, about 4% or less, about 3% or less, about 2% or less, or about 1% or less of total dietary calories.

In some embodiments of any aspect described above, the whole foods exclusionary diet is a nutritionally complete grain-free diet such as Specific Carbohydrate Diet™ (SCD™). The diet of the methods disclosed herein, such as SCD™, typically focuses on fresh, whole, unprocessed foods and has an overall low sugar and lactose content. To illustrate, the following tables summarize the foods that are allowed and not allowed in the SCD™ regimen. The tables are adapted from Gottschall, Elaine, Breaking the Vicious Cycle, The Kirkton Press, 1994, and the corresponding website http://www.breakingtheviciouscycle.info, incorporated herein by reference in their entireties. The whole foods exclusionary diet, such as SCD™, can be used along with standard medications or as “monotherapy”, without other medications.

| | |
|---|---|
| Allowed | Not Allowed |

MEATS & OTHER PROTEINS

| | |
|---|---|
| Fresh eggs of all kinds | Deli meats, processed meats, |
| Poultry (Chicken, Turkey, Quail) | smoked meats e.g. hotdogs, |
| Fish & Shellfish (Fresh or Frozen) | bologna, ham |
| Canned Fish (in water or oil) | Most bacons |
| Beef, Veal | Imitation shellfish |
| Pork | Most canned meats (e.g., Spam ®) |
| Lamb | Breaded meats |
| Collagen | Most seasoned, packaged meats |
| | Egg substitute products |
| | Meat substitutes (e.g., Tofutti ®) |

FRUITS

| | |
|---|---|
| Most fresh fruits | Plantain |
| Bananas with brown spots | Tamarind |
| Brown, ripened coconut | Green bananas or bananas without brown |
| Frozen fruits without additives | spots |
| Naturally dried fruit such as dates, raisins, | Green coconut meat and water |
| apricots without preservatives, flavors, or | Dried fruits with added sugar, flavors, or |
| sugar | corn syrup, e.g., most dried cranberries |
| Fruits canned in their own juices | Fruit juice made from concentrate (with |
| Certain brand dehydrated fruits and fruit | added sugar) |
| leathers (e.g., no added sugar) | Fruit juice with added corn syrup or sugar |
| | Fruit concentrates or fruit syrups |

VEGETABLES

| | |
|---|---|
| Most fresh, non-starchy vegetables | Canned vegetables (except pumpkin) |
| Frozen vegetables | Most canned tomato sauce, paste, purees |
| Some packaged tomato juices | Most commercial spaghetti or marinara |
| Some jarred tomato sauces | sauces |
| Homemade tomato juice, sauce, paste or | Root vegetables or tubers such as potatoes, |
| puree | yams/sweet potatoes, kohlrabi, parsnips, |
| French artichokes | yucca, taro, jerusalem artichokes, jicama |
| Canned pumpkin without additives | Starchy vegetables such as corn, baby |
| | corn, water chestnuts |
| | Lima beans, fava beans |
| | Soybeans, edamame, tofu |
| | Garbanzo beans, chickpeas |
| | Bean sprouts |
| | Mucilagenous vegetables such as okra, |
| | nopal, cactus, Aloe Vera |
| | Seaweeds, kelp, algae |
| | Bitter melon |
| | Plant meat substitutes |

FATS & OILS

| | |
|---|---|
| Butter | Margarine, butter substitutes |
| Ghee | Low fat and nonfat butters, cheeses |
| Oil of most variety including coconut, flax | Many commercial salad dressings |
| seed, olive, avocado, sunflower, safflower, | Most commercial spreads and dips such as |
| peanut | Best Foods ® |
| Mayonnaise (homemade or store bought if | |
| without starches, additives, or sugars) | |
| Olives: black, green, kalamata (read for | |
| illegal additives) | |
| Avocado | |
| Canola and corn oils * | |
| * Limit these highly processed oils. Use for | |
| deep frying only, <2 times per week | |

CHEESE AND OTHER DAIRY

| | |
|---|---|
| Butter | Unaged, typically soft cheeses, such as |
| Ghee | feta, chevre, ricotta, paneer, cream cheese, |
| Dry curd or Farmer's cheese | cottage cheese, fresh Mexican cheese |
| Yogurt cheese made with SCD yogurt | Processed cheeses, such as American, |
| Cheeses aged for >30 days, such as cheddar, | Velveeta, jarred or canned cheese, Kraft |
| Parmesan, Monterey Jack, Swiss, Havarti, | Singles |
| Brie, cotija | Sour cream, cream fraiche, any cream |
| | Cheese or cheese products that contain |
| | starch to prevent caking |

MILK/MILK SUBSTITUTES

| | |
|---|---|
| Homemade yogurt and yogurt products | Kefir |
| fermented for at least 24 hours | Commercial milk, cream, or yogurt of any |
| Homemade nut and seed milk | kind |
| Homemade or canned coconut milk/cream | Buttermilk |
| (read label carefully) | Dried milk solids |
| | Evaporated or condensed milk |
| | Commercial soy, rice, almond milk |
| | Commercial coconut milk beverages that |
| | have added starches, pectin, and stabilizers |

STARCH/BREAD/GRAINS

| | |
|---|---|
| Coconut flour | Wheat, semolina, barley, corn, rye, oats, |
| Lentil flour | rice, brown rice, buckwheat, amaranth, |
| Nut flours such as almond, pecan, hazelnut, | quinoa, millet, triticale, bulgar, spelt, |
| walnut | wheat berry, wheat germ, farro, cous cous, |
| | besan or gram flour, tapioca, banana flour, |
| | Any “ancient grains” or any flour, cereal, |
| | or other food made from these grains |
| | Starches of any kind such as arrowroot, |
| | tapioca, sago, potato, or cornstarch |
| | Chestnut and pea flour |

NUTS/SEEDS

| | |
|---|---|
| All fresh, raw, unprocessed, unflavored nuts | Chia, flax, hemp seeds |
| Most seeds | Processed or flavored nuts and seeds |
| Nut and seed butters made without additives | “Mixed Nuts” or “Salted Nuts” |
| | Nut and seed butters with additives (e.g., |
| | starch) or flavors |

LEGUMES & BEANS

| | |
|---|---|
| Lentils | Chickpeas, garbanzo beans |
| Navy, haricot, kidney, lima, black beans | Soybeans and edamame |
| Peas and split peas | Fava, faba, butter beans |
| Adzuki beans | Mung beans |
| * all beans must be soaked for 24 hours | Canellini beans |
| | Pinto beans |
| | Bean flour |
| | All canned beans |

HERBS/SEASONINGS/CONDIMENTS

| | |
|---|---|
| Salt and pepper | Fenugreek |
| All fresh herbs | Chicory root |
| Dried or dehydrated herbs without added | Seasoning mixes such as garlic salt, |
| sugar, starch or de-caker | seafood or steak mixes |
| Vinegar without added sugar, including | Bouillon cubes or instant soup bases |
| apple cider, malt, distilled, red wine, sherry, | Seasoning powders, i.e., onion or garlic |
| and rice vinegars | salt |
| | Premade sauces, such as ketchup, teriyaki, |
| | Worcestershire, etc. |
| | Soy sauce and Tamari |
| | Fish sauce |
| | Gourmet mustards (due to added sugars) |
| | Balsamic vinegar |

SWEETENERS

| | |
|---|---|
| Honey | Granulated, brown, cane, or table sugar |
| Saccharin | Coconut or palm sugar, turbinado |
| Pure fruit juice | Molasses |
| Dates | Maple syrup and agave syrup |
| | Stevia |
| | Splenda (sucralose) |
| | Sugar alcohols such as sorbitol, mannitol, |
| | xylitol, maltitol |

BEVERAGES

| | |
|---|---|
| Water | Beer, wine, hard cider |
| Pure fruit juice | Sherry, cordials, liqueurs, brandy, port, |
| Carbonated water and club soda | sake |
| Herb teas such as chamomile, peppermint, | Sport and energy drinks |
| hibiscus | Pedialyte ® |
| Weak coffee | Kombucha |
| Weak black tea | Water or milk kefir |
| | Coffee mixes with sugar, |

OTHER

| | |
|---|---|
| Unflavored gelatin | Coconut Aminos |
| Baking Soda | Pectin |
| Almond and vanilla extracts (without | Thickeners such as potato, sago, corn, |
| alcohol) | arrowroot |
| | Baking powder |
| | Guar gum, xanthan gum |
| | Carob |
| | Cocoa/chocolates |
| | Jello mixes |
| | Yeast |
| | Licorice |
| | Miso |

Thus, in some embodiments, the compositions disclosed herein, such as SCD™, can optionally comprise one or more of the following: meats without additives, poultry, fish shellfish and eggs; legumes, including dried navy beans, lentils, peas, split peas, unroasted cashews and peanuts in a shell, all-natural peanut butter and lima beans; dairy limited to cheeses such as cheddar, Colby, Swiss, dry curd cottage cheese; and homemade yogurt fermented for at least 24 hours; most fresh, frozen, raw or cooked vegetables and string beans; fresh, raw or cooked, frozen or dried fruits with no added sugar; most nuts and nut flours; most oils, teas, coffee, mustard, cider or white vinegar and juices with no additives or sugars; honey.

In some embodiments of the methods and compositions disclosed herein, the whole foods exclusionary diet, such as SCD™, lacks or has minimal amounts of the following components: sugar, molasses, maple syrup, sucrose, processed fructose, including high-fructose corn syrup or any processed sugar; all grains including corn, wheat, wheat germ, barley, oats, rice and others. This includes bread, pasta and baked goods made with grain-based flour; canned vegetables with added ingredients; some legumes; seaweed and seaweed byproducts; starchy tubers such as potatoes, sweet potatoes, and turnips; canned and most processed meats; canola oil and commercial mayonnaise; all milk and milk products high in lactose such as mild cheddar, commercial yogurt, cream and sour cream, and ice cream; candy, chocolate and products that contain FOS (fructooligosaccharides)

In some embodiments of the methods and compositions disclosed herein, the whole foods exclusionary diet lacks or has minimal amount of calories derived from one or more of the following: added sugars, molasses, maple syrup, agave syrup, sucrose, processed fructose, high-fructose corn syrup, grains, lactose, starch derived from tubers, pectin, sugar alcohols, and fructooligosaccharides. As used herein, “added sugars” refers to simple carbohydrates, such as sucrose and fructose, that are not naturally present in foods such as vegetables, fruits, and berries.

In some embodiments of any aspect described above, the Clostridium is Clostridium difficile.

In some embodiments of any aspect described above, the subject is a mammal, such as a rodent, human, or non-human primate. In some embodiments of any aspect described above, the subject is a human. In some embodiments, the subject is a human child 18 years old or less. In some embodiments, the subject is an adult human. In some embodiments, the subject has been previously treated with antibiotics prior to the administration of the diet the disclosure. In some embodiments, the subject has been previously treated with antibiotics effective against Clostridium, such as Clostridium difficile.

In some embodiments of any aspect described above, the modifying the subject's diet to remove all (or substantially decrease) intake of grains, milk products (except for hard cheeses and yogurt fermented at least 24 hours), and sweeteners, such as added sugars, (except for honey whole) (e.g., the whole foods exclusionary diet such as SCD™) is administered for an effective time period. In some embodiments, the effective time period is a time period ranging between about 1 week and about 12 weeks, such about 1 week and about 8 weeks, about 1 week and about 6 weeks, and about 2 weeks and about 6 weeks least (with end points inclusive in the range). In some embodiments, the effective time period is at least about 1 week, at least about 2 weeks, at least about 3 weeks, at least about 4 weeks, at least about 5 weeks, at least about 6 weeks, at least about 7 weeks, at least about 8 weeks, at least about 9 weeks, or at least at least about 10 weeks. The term “about,” when preceding a time period in weeks, refers to an acceptable variation of 1/7 up or down of the referred time period. In some embodiments, the modifying the subject's diet to remove all (or substantially decrease) intake of grains, milk products (except for hard cheeses and yogurt fermented at least 24 hours), and sweeteners (except for honey whole) (e.g., the whole foods exclusionary diet such as SCD™) is administered for a time period of about 1 week, about 2 weeks, about 3 weeks, about 4 weeks, about 5 weeks, about 6 weeks, about 7 weeks, about 8 weeks, about 9 weeks, and about 10 weeks.

In some embodiments, the step of modifying the subject's diet to remove all (or substantially decrease) intake of grains, milk products (except for hard cheeses and yogurt fermented at least 24 hours), and sweeteners (except for honey whole) (e.g., the administering of the whole foods exclusionary diet such as SCD™) is followed by administering of a healthy diet that simply reduces, i.e., removes excess, sugars (such as added sugars), milk, and processed foods. Such a diet can be similar to a Mediterranean diet as understood in the art.

The ameliorative effects of the methods disclosed herein can be detected as early as 2 to 4 weeks at the onset of the diet modification and can last up to 12 weeks or more, using any suitable detection method, for example, PCR analysis as described above.

The references cited herein are incorporated by reference. Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

As can be appreciated from the disclosure above, the present invention has a wide variety of applications. The invention is further illustrated by the following examples, which are only illustrative and are not intended to limit the definition and scope of the invention in any way.

### Examples

The following describes a study entitled “Dietary Therapy for Clostridium Difficile Colonization: A case series.”

Introduction

With the importance of the intestinal microbiota in development of C. difficile and with the known impact of diet on the intestinal microbiota, we report 3 patients with colonization/recurrence of C. difficile who experienced reduction or eradication of C. difficile by dietary modification.

Materials and Methods

Disclosed herein is a prospective review of two patients in an open-label study designed to determine tolerability, preliminary safety, and potential efficacy of the specific carbohydrate diet (SCD™) in pediatric patients with inflammatory bowel disease (IBD) as well as a retrospective case of recurrent C. difficile. The SCD™ diet is a diet which removes all grains, milk products except for hard cheeses and yogurt fermented ×24 hours and sweeteners except for honey. It also removes the vast majority of food additives.

The prospective study was approved by the Institutional Review Board of Seattle Children's Hospital. All patients/participants provided written informed consent or assent. The prospective study was registered with ClinicalTrials.gov (number: NCT02213835). Study participants were recruited from Seattle Children's Hospital. For the prospective study patients with Crohn's disease (CD) or ulcerative colitis (UC) ages 8-21 with mild or moderate disease activity (based on PUCAI or PCDAI scores) were eligible to be enrolled. Prior to the study no change in medication(s) for IBD could occur for a minimum of one month for immunosuppressive medications and two months for biological medications. Patient stool was analyzed for stool microbiome.

Results

Patient one is a 13-year-old male with ileal Crohn's disease not responsive to standard medical therapy who was currently on ustekinumab. He presented with abdominal pain, anemia, and elevated inflammatory markers including stool calprotectin. At the time of enrollment in the SCD™ study he was having two semi formed stools per day. His initial pediatric PCDAI was 40 indicating moderate disease. He initiated the SCD diet and had a single follow-up 2 weeks later. At that visit, he discontinued the study secondary to difficulty maintaining the diet and little clinical change. His PCDAI at last study visit was 37.5 (moderate disease activity) and there was little change in his inflammatory markers. Stool microbiome analysis revealed a decrease in relative abundance of C. difficile from 8.5 to 6.7%.

Patient two is a 13-year-old female with history of ileocolonic Crohn's disease who presented with a flare of her Crohn's disease despite maintenance therapy with methotrexate. Her presenting symptoms were abdominal pain, anemia and elevated inflammatory markers including stool calprotectin. She was having two soft formed stools per day with traces of blood on stool. She initiated the SCD™ diet and had followed up at 2, 4, 8 and 12 weeks. She clinically responded to the diet with a PCDAI decreasing from 25 to 0. Her baseline microbiome analysis showed a relative abundance of C. difficile at 0.7% which decreased to 0% at 2 and 12 weeks post initiation of diet.

The third patient is a 12-year-old female who received two hematopoietic stem cell transplants (HSCT) for Acute Myelogenous Leukemia. She also had a history of end-stage renal disease requiring hemodialysis related to thrombotic microangiopathy, history of CMV colitis, and graft versus host disease. She was gastrostomy tube dependent and received Vivonex TEN and Novasource Renal as nutritional supplement. She also ate a small amount of food by mouth. Two years following HSCT, she developed her first C. difficile infection (CDI). She was initially treated with metronidazole but subsequently developed 14 documented recurrences during which she received courses of metronidazole, oral vancomycin, oral vancomycin with a prolonged taper, and fidaxomicin. She received 6 fecal microbial transplants with varying degrees of short-lived relief from diarrheal symptoms Immunosuppression at the time of first FMT included tacrolimus and prednisolone. She tolerated each FMT without infection or adverse event.

Following her final recurrent CDI, it was decided to treat her with a 14-day course of vancomycin and then proceed with dietary therapy, specifically a modified blenderized SCD™ made to address her renal disease. She maintained on the SCD™ diet for over 2 months, and then added in non-SCD™ foods including whole grains. After 4 months she transitioned to a whole foods diet in conjunction with Nourish formula, a whole foods formula. She has not had a reoccurrence of CDI for over 2 years. Initial analysis of her intestinal microbiome done one week after vancomycin therapy showed a profound dysbiosis with only 2 organisms making up over 98% of the bacteria. Subsequent analysis one month later on the SCD™ showed a significant increase of biodiversity with no evidence of C. difficile. While the patient stayed asymptomatic, C. difficile colonization did recur in the intestinal microbiome by microbiome analysis at 6 months after discontinuation of the SCD™. She remained asymptomatic and C. difficile toxin and antigen negative for over two years since dietary change.

Discussion

While classic risk factors for the development of C. difficile include antibiotic use, malignancy, inflammatory bowel disease, organ transplant and cystic fibrosis, diet is also likely to have an impact through its effect on the intestinal microflora. Diet has a profound impact on the intestinal microbiome. Diet has been shown to change the intestinal microbiome in reproducible ways. In an animal study of genetically alter mice, diet shaped the intestinal microbiome in similar ways across genotypically different hosts. In humans, diet has been shown to change the intestinal microbiome rapidly with changes sustained as long as dietary changes were sustained. In addition, commercial formulas have been shown to decrease the biodiversity of the intestinal microbiome as well as increase the incidence of C. difficile colonization in the gastrointestinal tract.

Prior experience with dietary therapy in active disease initially focused on the SCD™ in IBD. Diet not only had clinical efficacy in IBD therapy but was also associated with dramatic shifts in the intestinal microbiome. C. difficile colonization, which was noted in 2 of our patients, is known to be more common in patients with IBD. The two patients noted in this case series had no evidence of C. difficile colitis, i.e. no diarrheal symptoms and stool antigen negative, which would suggest C. difficile was colonized and not an active infection. With dietary changes alone one patient had decreased C. difficile colonization in the microbiota while the other had resolution of C. difficile in the microbiota.

With evidence that C. difficile colonization could be resolved with dietary changes, we propose a therapeutic dietary change for a patient with recalcitrant C. difficile. While one patient had many potential risk factors for the development of C. difficile including chronic antibiotic use, renal failure, history of bone marrow transplant and poor dietary intake, by changing her diet, she had both a dramatic clinical improvement as well as shift in intestinal microbiome. Initial analysis of her intestinal microbiome done one week after vancomycin therapy showed a profound dysbiosis with only 2 organisms making up over 98% of the bacteria. This would have likely led to another re-occurrence of C. difficile infection. With dietary change, she has remained asymptomatic and C. difficile toxin and antigen negative for over two years.

The mechanism of action of dietary change on C. difficile colonization could include removal of food additives, decreased sugars, and increase in fiber content. A high fiber diet was shown to play a role in eradicating C. difficile from the intestinal microbiome in one dietary animal model. In addition, certain foods and/or additives may allow C. difficile to grow more efficiently and therefore play a role in colonization of C. difficile.

### CONCLUSIONS

This disclosure is the first to demonstrate that diet has an impact on C. difficile in patients with colonization or recurrent infections. Given the known impact of the intestinal microbiome on the proliferation of C. difficile and the known impact of diet on the intestinal microbiome, a potential mechanism of action for dietary therapy exists for the clearance of C. difficile in these patients. The implications of diet's effect on disease occurrence and reoccurrence could profoundly change the disease landscape.

While illustrative embodiments have been illustrated and described, it will be appreciated that various changes can be made therein without departing from the spirit and scope of the invention.

1. A method of treating intestinal dysbiosis associated with Clostridium infection in a subject, comprising administering to the subject in need thereof a whole foods exclusionary diet for an effective time, wherein the whole foods exclusionary diet is a diet that excludes grains, sugars other than honey, and milk products other than hard cheeses and yogurt fermented for greater than 24 hours.

2. A method of preventing Clostridium colonization or Clostridium recolonization in the intestines of a subject, comprising administering to the subject in need thereof a whole foods exclusionary diet for an effective time, wherein the whole foods exclusionary diet is a diet that excludes grains, sugars other than honey, and milk products other than hard cheeses and yogurt fermented for greater than 24 hours.

3. A method of reducing or inhibiting Clostridium infection in a subject, comprising administering to the subject in need thereof a whole foods exclusionary diet for an effective time, wherein the whole foods exclusionary diet is a diet that excludes grains, sugars other than honey, and milk products other than hard cheeses and yogurt fermented for greater than 24 hours.

4. The method of any one of claims 1-3, wherein the Clostridium is Clostridium difficile.

5. The method of any one of claims 1-4, wherein the subject is a mammal.

6. The method of any claim 5, wherein the mammal is a human.

7. The method of claim 6, wherein the subject is a human child 18 years old or less.

8. The method of claim 6, wherein the subject is an adult human.

9. The method of any one of claims 1-3, wherein the whole foods exclusionary diet is administered for at least one week, at least two weeks, at least three weeks, at least 1 month, at least 2 months, or at least 3 months.

10. The method of any one of claims 1-3, wherein the effective time of administration of the whole foods exclusionary diet is at least one week, at least two weeks, at least three weeks, at least 1 month, at least 2 months, or at least 3 months.

11. The method of any one of claims 1-3, wherein the subject has been treated with one or more antibiotics effective against Clostridium prior to administration of the whole foods exclusionary diet.

12. The method of any one of the preceding claims, wherein the whole foods exclusionary diet is a nutritionally complete grain-free diet with low sugar and lactose content.

13. The method of any one of the preceding claims, wherein the whole foods exclusionary diet is a nutritionally complete diet that excludes grains, grain products, milk products other than 24-hour fermented homemade yogurt and cheeses aged greater than 30 days, starchy vegetables, root vegetables, processed foods, and added sweeteners other than honey.

14. The method of any one of the preceding claims, wherein the whole foods exclusionary diet is Specific Carbohydrate Diet.

15. The method of any one of the preceding claims, wherein the whole foods exclusionary diet comprises one or more of the following:
meats without additives, poultry, fish, shellfish, eggs; legumes, including dried navy beans, lentils, peas, split peas, unroasted cashews and peanuts in a shell, all-natural peanut butter, lima beans; hard cheeses aged more than 30 days, homemade yogurt fermented for at least 24 hours; fresh, frozen, non-starchy vegetables, string beans, whole fruits, whole nuts, whole nut flours, vegetable oils, teas, coffee, mustard, cider vinegar, white vinegar, juices with no additives, and honey.

16. The method of any one of the preceding claims, wherein the whole foods exclusionary diet lacks or has minimal amount of calories derived from one or more of the following:
added sugars, molasses, maple syrup, agave syrup, sucrose, processed fructose, high-fructose corn syrup, grains, lactose, starch derived from tubers, pectin, sugar alcohols, and fructooligosaccharides.

17. The method of any one of the preceding claims, wherein the whole foods exclusionary diet lacks or has minimal amount of calories derived from one or more of the following:
corn, wheat, wheat germ, barley, oats, rice, bread, pasta, baked goods made with grain-based flour; seaweed, seaweed byproducts, potatoes, sweet potatoes, turnips; canned meats, processed meats, canola oil, commercial mayonnaise; milk, milk products high in lactose, soft cheese, commercial yogurt, cream, sour cream, ice cream, candy, and chocolate.

18. The method of claim 16 or claim 17, wherein the minimal amount of calories is about 5% or less, about 4% or less, about 3% or less, about 2% or less, or about 1% or less of total dietary calories.
